(19) United States
(12) Patent Application Publication
(10) Pub. No.: US 2017/0292161 A1
(43) Pub. Date: Oct. 12, 2017

(54) METHODS AND COMPOSITIONS FOR DETECTING COLORECTAL NEOPLASIAS USING MICRO RNAs

(71) Applicant: University of Louisville Research Foundation, Inc., Louisville, KY (US)

(72) Inventors: Susan Galandiuk, Louisville, KY (US); Robert Eichenberger, Louisville, KY (US); Henry Roberts, Louisville, KY (US)

(21) Appl. No.: 15/481,072

(22) Filed: Apr. 6, 2017

Related U.S. Application Data
(60) Provisional application No. 62/319,003, filed on Apr. 6, 2016.

(51) Int. Cl. C12Q 1/68

(52) U.S. Cl. CPC C12Q 1/6886; C12Q 2600/178; C12Q 2600/158

(57) ABSTRACT

Methods of detecting or predicting the presence of colorectal neoplasia are described based on the amounts of particular miRNAs in plasma.

### CROSS-REFERENCE TO RELATED APPLICATIONS

This application claims priority to U.S. Provisional Patent Application No. 62/319,003, filed on Apr. 6, 2016, which is herein incorporated by reference in its entirety.

### TECHNICAL FIELD

This disclosure generally relates to microRNA (miRNA) and its use in screening and detection of colorectal neoplasia.

### BACKGROUND

Currently, colorectal cancers (CRCs) are primarily diagnosed through colonoscopy and flexible sigmoidoscopy, procedures that are expensive, require bowel preparation, sedation, and may also be associated with medical complications. Many patients hesitate or completely avoid having these procedures due to any or all of the above. Less-invasive diagnostic methods such as fecal occult blood tests and DNA-based stool tests require viable stool specimens, and plasma-based assays to date have exhibited a lack of sensitivity and specificity.

70% of rectal cancer patients exhibit pathological down-staging following neoadjuvant chemoradiation. 15% to 20% of individuals will exhibit a complete response, while 30% to 50% of individuals undergoing curative resection will have a recurrence. There are no reliable markers to monitor an individual's response to treatment; currently, PET, MRI, CT, ERUS, and CEA assays are used, which can be inaccurate, expensive, and subject to inter-observer variation.

In addition, colorectal neoplasms (colorectal cancer (CRC) and colorectal advanced adenoma (CAA)) frequently develop in individuals at ages when other common cancers also occur. Current screening methods lack sensitivity, specificity, and have poor patient compliance.

Therefore, a need exists for a new, minimally invasive, inexpensive test to reliably detect and distinguish colorectal neoplasms from other types of cancers, and monitor an individual's response to treatment of CRC.

### SUMMARY

The present disclosure describes methods of detecting or predicting the presence of colorectal neoplasia based on the amounts of particular miRNAs in plasma.

A plasma-based screening test is described that differentiates individuals with colorectal cancer and colorectal advanced adenomas from individuals with other cancers with high sensitivity and specificity. The plasma-based screening test described herein also differentiates individuals with colorectal cancer and colorectal advanced adenomas from individuals without cancer with high sensitivity and specificity. Since there is currently no accurate diagnostic blood test for colorectal neoplasia, the methods described herein have profound, immediate clinical implications.

In one aspect, a method of detecting an individual with colorectal advanced adenoma (CAA) and/or colorectal cancer (CRC) is provided. Such a method typically includes providing a biological sample from an individual prior to treatment for CAA or CRC, wherein the biological sample is blood or plasma; providing a biological sample from the individual following treatment for CAA or CRC, wherein the biological sample is blood or plasma; and determining the levels of miR-21, miR-29c, miR-122, miR-192, miR-346, miR-372 and miR-374 in the biological sample using RNU6 and/or miR-520d-5p as internal reference(s). Generally, an increase or decrease in each miRNA, as described in the attached document, is indicative of CAA and/or CRC in the individual.

In another aspect, a method of monitoring an individual receiving treatment for colorectal advanced adenoma (CAA) and/or colorectal cancer (CRC) is provided. Such a method typically includes providing a biological sample from an individual prior to treatment for CAA or CRC, wherein the biological sample is blood or plasma; providing a biological sample from the individual following treatment for CAA or CRC, wherein the biological sample is blood or plasma; and comparing the levels of miR-21, miR-29c, miR-122, miR-192, miR-346, miR-372 and miR-374a in the biological sample from the individual after treatment with the levels in the biological sample from the individual prior to treatment using RNU6 and/or miR-520d-5p as internal reference. Generally, the levels of miR-21302a, miR-29c, miR-122, miR-192, miR-346, miR-372 and miR-374a in the biological sample from the individual after treatment relative to the levels of miR-21302a, miR-29c, miR-122, miR-192, miR-346, miR-372 and miR-374a in the biological sample from the individual prior to treatment indicates, as described in the attached document, whether or not the individual undergoes continued or further treatment and what that treatment should be.

In some embodiments, the level of miRNAs is determined using RT-PCR. In some embodiments, the treatment includes neoadjuvant chemoradiation. In some embodiments, the treatment includes endoscopy or surgery. In some embodiments, the further treatment includes chemotherapy. In some embodiments, the further treatment includes radiation. In some embodiments, the further treatment includes endoscopy or surgery.

In yet another aspect, an article of manufacture is provided. Such an article of manufacture typically includes a pair of miR-21 amplification primers; a pair of miR-29c amplification primers; a pair of miR-122 amplification primers; a pair of miR-192 amplification primers; a pair of miR-346 amplification primers; a pair of miR-372 amplification primers; and a pair of miR-374a amplification primers. Representative sequences of such pairs of primers are shown in the attached document.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing the study outline. Colorectal cancer (CRC), colorectal advanced adenoma (CAA), breast cancer (BC), lung cancer (LC) and pancreatic cancer (PC).

FIG. 2A shows ROC curves and AUC for the panel of miR-21, miR-29c, miR-346 and miR-374a for CRC, CAA, BC, LC and PC vs control in the validation data set.

FIG. 2B shows ROC curves and AUC for the panel of miR-21, miR-29c, miR-372 and miR-374a for CAA and CRC vs BC, LC and PC in the test data set.

FIG. 2C shows ROC curves and AUC for the panel of miR-29c, miR-122, miR-192 and miR-374a for CRC vs CAA in the test data set.

FIG. 3A are graphs showing the prediction scores for comparing CRC, CAA, BC, LC, and PC vs. Control in the Validation cohort.

FIG. 3B are graphs showing the prediction scores for comparing CAA and CRC vs. BC, LC and PC in the Test cohort.

FIG. 3C are graphs showing the prediction scores for comparing CAA vs. CRC in the Test cohort.

FIG. 3D are graphs showing the prediction scores for comparing CAA and CRC vs. Control in the Test cohort.

FIG. 3E are graphs showing the prediction scores for comparing CRC vs. Control in the Test cohort.

FIG. 3F are graphs showing the prediction scores for comparing CAA vs. Control in the Test cohort.

### DETAILED DESCRIPTION

A panel of particular microRNAs is described herein that is very sensitive for detecting individuals with CAA or CRC, and can be used to monitor treatment response in those individuals. The methods and materials described herein provide less invasive biomarkers for monitoring disease recurrence, which has significant implications, particularly in today's era of watchful waiting following neoadjuvant therapy for rectal cancer.

### Methods of Detecting Colorectal Neoplasia

A miRNA panel has been developed and is described herein that can differentiate colorectal neoplasia from controls and other common cancers with high sensitivity and specificity. The miRNA panel described herein also is able to accurately distinguish CRC from its precursor lesion, CAA. There is a vital need for a non-invasive, reliable and cost-effective tool to diagnose CRC or CAA.

Methods of screening an individual for the presence or absence of colorectal cancer (CRC, which includes early-stage CRC) or colorectal advanced adenoma (CAA) are described herein. Typically, a biological sample of blood or plasma is provided from the individual and the levels of the indicated miRNAs are determined. In some instances, a biological sample of blood or plasma is provided from the individual prior to and after receiving treatment, and the levels of the indicated miRNAs are determined and compared between samples. Comparing the indicated miRNAs before and after treatment (e.g., neoadjuvant chemoradiation, endoscopy or surgery) can be used to help determine whether or not the individual undergoes further treatment and what that treatment might include (e.g., chemotherapy, radiation, endoscopy or surgery).

Methods of determining the level of one or more miRNAs are well known in the art. For example, the level of miRNAs can be determined using routine RT-PCR. Simply by way of example, total RNA can be extracted from raw serum (e.g., 0.5 mL of raw serum) using miRNeasy (Qiagen, Venlo, Limburg), reverse transcribed and amplified in the presence of an appropriate pair of primers using, for example, a RT-PCR Kit from Life Technologies (Carlsbad, Calif.), to produce cDNAs. Following RT-PCR, the amount or level of the resulting amplified product can be determined using, for example, a spectrophotometer (e.g., a Nanodrop 2000 from Thermo Scientific® (Middlesex, Mass.)). Alternatively, other methods suitable for detecting and quantitating RNAs can be used such as Northern blotting following by phosphoimaging.

The miRNA panel described herein offers a new method of blood-based screening for detection of colorectal cancer and its precursor lesion, CAA. Using the diagnostic algorithms provided herein, colorectal neoplasia can be distinguished from other cancers with an AUC of 0.79, and CRC can be differentiated from CAA with an AUC of 0.98. The ability to detect advanced colorectal adenoma with such high sensitivity and specificity is unique. Also, the ability to distinguish subjects with other cancers (breast, lung, and pancreas), which is more representative of the population as a whole, provides a number of advantages over existing tests or assays. In addition, a predictive model that distinguishes CRC from CAA has not previously been described. Further, a method that allows for a patient's sample to be tested without the need for comparison to an external control (e.g., a “normal” patient sample) provides several advantages in the laboratory setting.

Data reproducibility is crucial for any potential diagnostic test. Previous studies examined the effects of time on plasma extraction, method of RNA extraction, as well as issues of inter- and intra-operator variability. In these studies, it was determined that rapid plasma extraction (<12 h) yields optimal results, as does the use of a modified phenol/guanidine-based lysis and silica membrane-based RNA purification technique for RNA extraction (Rice et al., 2015, PLoS One, 10(4):e0121948).

Reference genes, and particularly endogenous reference genes, are critical for standardizing data acquisition and reliable reporting of results. The ideal endogenous reference gene should have medium to high levels of expression to be able to consistently measure the level of expression in all plasma samples. A combination of U6 small nuclear-1 (RNU6) [GenBank Accession No. NR_004394.1] and miR-520d-5p [GenBank Accession No. NR_030204.1] was used as the endogenous reference genes in the methods described herein.

Although not intended to be limiting, individuals that might benefit from the methods disclosed herein include those individuals that are at a higher risk for CRC (e.g., individuals greater than 50 years old, smokers, African-Americans, and/or individuals that eat a diet low in fiber), individuals that have a personal history of CRC or polyps, or individuals that have a family history of sporadic CRC or polyps.

The plasma-based miRNA panel described herein may have other potential uses such as monitoring therapy and predicting treatment response. CAA and CRC patients can be prospectively monitored over time and miRNA expression profiles compared in samples obtained prior to and after treatment (e.g., endoscopic or surgical treatment) to determine and/or identify those plasma-based biomarkers that revert to “normal” levels after treatment (e.g., lesion removal by endoscopic or surgical therapy).

The methods and miRNA markers described herein resulted in very high sensitivity and specificity values for predicting or detecting the presence or absence of CRC in an individual. The methods and miRNA markers described herein accurately differentiates patients with colorectal neoplasia from those with other cancers, or controls, using plasma. In addition, the methods and miRNA markers described herein differentiates between patients with CRC and patients with CAA. This has significant implications for development of a non-invasive, reliable and reproducible screening test for the detection of colorectal neoplasia, which would be superior to current non-invasive screening methods.

### Nucleic Acids and Methods Associated Therewith

microRNAs, also referred to as miRNAs or miRs, are short, non-protein encoding RNA molecules that bind to complementary sequences on target mRNAs and affect gene expression. miRNAs are useful due to their inherent stability and their involvement in numerous regulatory processes. In addition, certain miRNAs have been shown to be sensitive and specific biomarkers for certain diseases. As described herein, a particular combination of miRNAs, miR-21, miR-29c, miR-122, miR-192, miR-346, miR-372 and miR374a, has been found to be highly predictive and distinguishable for colorectal neoplasia.

The sequence of miR-21 from Homo sapiens can be found in GenBank Accession No. NR_029493.1; the sequence of miR-29c from Homo sapiens can be found in GenBank Accession No. NR_029832.1; the sequence of miR-122 from Homo sapiens can be found in GenBank Accession No. NR_029667.1, the sequence of miR-192 from Homo sapiens can be found in GenBank Accession No. NR_029578.1, the sequence of miR-346 from Homo sapiens can be found in GenBank Accession No. NR_029907, the sequence of miR-372 from Homo sapiens can be found in GenBank Accession No. NR_029865.1, and the sequence of miR-374a from Homo sapiens can be found in GenBank Accession No. NR_030785.1. Each of these sequences are incorporated herein by reference in their entirety.

Depending upon the application, a nucleic acid can be used that does not have the exact sequence shown in the GenBank Accession Nos. referred to above. For example, a miR-21 nucleic acid can be used that has at least 95% sequence identity (e.g., at least 96%, 97%, 98% or 99% sequence identity) to the sequence shown in GenBank Accession No. NR_029493.1. Similarly, a miR-29c nucleic acid can be used that has at least 95% sequence identity (e.g., at least 96%, 97%, 98% or 99% sequence identity) to the sequence shown in GenBank Accession No. NR_029832.1; a miR-122 nucleic acid can be used that has at least 95% sequence identity (e.g., at least 96%, 97%, 98% or 99% sequence identity) to the sequence shown in GenBank Accession No. NR_029667.1; a miR-192 nucleic acid can be used that has at least 95% sequence identity (e.g., at least 96%, 97%, 98% or 99% sequence identity) to the sequence shown in GenBank Accession No. NR_029578.1; a miR-346 nucleic acid can be used that has at least 95% sequence identity (e.g., at least 96%, 97%, 98% or 99% sequence identity) to the sequence shown in GenBank Accession No. NR_029907; a miR-372 nucleic acid can be used that has at least 95% sequence identity (e.g., at least 96%, 97%, 98% or 99% sequence identity) to the sequence shown in GenBank Accession No. NR_029865.1; and/or a miR-374a nucleic acid can be used that has at least 95% sequence identity (e.g., at least 96%, 97%, 98% or 99% sequence identity) to the sequence shown in GenBank Accession No. NR_030785.1.

To determine or calculate percent sequence identity between two sequences, the two sequences are aligned and the number of identical matches (of nucleotides or amino acid residues) between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (Basic Local Alignment Search Tool) programs, available at ncbi.nlm.nih.gov on the World Wide Web. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

Nucleic acids that differ in sequence from GenBank Accession Nos. NR_029493.1; NR_029832.1; NR_029667.1; NR_029578.1; NR_029907; NR_029865.1; NR_030785.1 can be obtained using any number of methods. For example, changes can be introduced into nucleic acid sequences using mutagenesis (e.g., site-directed mutagenesis, PCR-mediated mutagenesis) or by chemically synthesizing a nucleic acid molecule having such changes.

Oligonucleotide primers for use in detecting and quantitating the indicated miRNAs (e.g., for use in RT-PCR reactions) can be readily designed by a person skilled in the art. Oligonucleotide primers can be designed using, for example, a computer program such as Vector NTI® (Life Technologies); Primer Premier (Premier Biosoft); OLIGO (Molecular Biology Insights) and the sequence of the miRNA. Typically, oligonucleotide primers are 10 to 30 or 40 or 50 nucleotides in length (e.g., 10, 11, 12, 13, 14, 15, 16, 17, 18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), but can be longer or shorter if appropriate amplification conditions are used. Oligonucleotides can be obtained by restriction enzyme digestion of a nucleic acid molecule or can be prepared by standard chemical synthesis and other known techniques.

Nucleic acids (e.g., miRNAs) can be isolated. As used herein, an “isolated” nucleic acid is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Vectors also are provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6xHis tag, glutathione S-transferase (GST)).

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny or potential progeny of such a cell. A host cell can be any prokaryotic or eukaryotic cell. For example, nucleic acids can be replicated and, if desired, expressed, in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188) with an appropriate pair of oligonucleotides (e.g., primers). A number of modifications or variations to the original PCR have been developed and also can be used to detect a nucleic acid. Such modifications or variations to the original PCR are well known to the skilled artisan.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. In one embodiment, “high stringency” hybridization conditions refer to washes that include 5-6x SSC, 0.1-0.5% SDS, and 50% formamide at 65° C.

Detection (e.g., of an amplification product or a hybridization complex) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that, although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, Calif.).

### Articles of Manufacture or Kits

Articles of manufacture are provided that can be used to detect CRC or CAA in an individual. An article of manufacture as provided herein can include one or more nucleic acids (e.g., primers and/or probes) for detecting and determining the amount or level of miR-21, miR-29c, miR-122, miR-192, miR-346, miR-372 and miR374a, together with suitable packaging materials.

Articles of manufacture provided herein also can include one or more detectable labels (e.g., one or more fluorescent moieties). For example, an article of manufacture may include a different detectable label for identifying and distinguishing each of the seven miRNAs used in the methods described herein. Examples of suitable detectable labels are well known in the art.

Articles of manufacture provided herein also can contain a package insert or package label having instructions thereon for using such nucleic acids to determine the amount of the corresponding miRNA in a sample. Articles of manufacture may additionally include reagents for carrying out the methods disclosed herein (e.g., buffers, enzymes, or co-factors). Such reagents may be specific for one of the commercially available PCR platforms.

### Treatments for Colorectal Neoplasia

As described herein, particular miRNA profiles have been correlated with an increased risk of developing or having colorectal neoplasia. Thus, the methods described herein also can include selecting a treatment regimen for a subject determined to be at risk for developing colorectal neoplasia. The methods described herein also can include administering a treatment regimen to a subject having, or at risk for developing colorectal neoplasia to thereby treat, prevent or delay further progression of the disease.

Colorectal neoplasia typically is treated using any number of treatment options: watchful waiting, which includes closely monitoring the individual's condition in the absence of treatment unless symptoms appear or change; surgery (e.g., radical prostatectomy, lymphadenectomy, transurethral resection of the prostate (TURP), orchidectomy, cryosurgery); radiation therapy (e.g., external or internal); hormone therapy (e.g., with luteinizing hormone releasing hormone (LHRH) agonists, antiandrogens, and estrogens); chemotherapy; high-intensity focused ultrasound; and/or therapy with one or more biologics (e.g., therapeutic antibodies).

As used herein, the term “treat” or “treatment” is defined as the application or administration of a treatment regimen (e.g., a therapeutic agent or modality) to an individual (e.g., a patient). The individual can have colorectal neoplasia, a symptom of colorectal neoplasia, or be at risk of developing colorectal neoplasia (i.e., having one or more of the risk factors for colorectal neoplasia known in the art or described herein). The goal of treatment can be to cure, heal, alleviate, relieve, alter, remedy, ameliorate, palliate, or improve colorectal neoplasia, the symptoms of colorectal neoplasia or the predisposition toward colorectal neoplasia.

The methods described herein can further include the step of monitoring the individual, e.g., for a change (e.g., an increase or decrease) in one or more of the diagnostic criteria for colorectal neoplasia listed herein, or any other parameter related to clinical outcome. The individual can be monitored during one or more of the following periods: prior to beginning of treatment; during treatment; or after one or more elements of the treatment have been administered. Monitoring can be used to evaluate the need for further treatment with the same or a different therapeutic agent or modality.

The methods described herein can be used, for example, to evaluate the suitability of, or to choose between, alternative treatments (e.g., a particular dosage, mode of delivery, time of delivery, and/or inclusion of adjunctive therapy (e.g., administration in combination with a second agent)). In a preferred embodiment, a treatment for colorectal neoplasia can be evaluated by administering the same treatment or combinations or treatments to a subject having colorectal neoplasia and a genetic profile as described herein and to a subject that has colorectal neoplasia but does not have a genetic profile as described herein.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### General Methods

The study described herein was approved by the University of Louisville Institutional Review Board, and written informed consent was obtained from all patients. The study population consisted of consecutive patients recruited from a large University colon and rectal surgery practice (n=110) and patients derived from the University of Louisville Surgical Biorepository (n=220).

As explained in more detail below, plasma was screened for 380 miRNAs using microfluidic array technology from a “Training” cohort of 60 patients: 10 each control, CRC, CAA, breast cancer (BC), pancreatic cancer (PC) and lung cancer (LC). Uniquely dysregulated miRNAs specific for colorectal neoplasia (p<0.05, false discovery rate: 5%, adjusted alpha=0.0038) were identified. These miRNAs were evaluated using single assays in a “Test” cohort of 120 patients. A mathematical model was developed to predict blinded sample identity in a 150 patient “Validation” cohort using repeat-sub-sampling validation of the testing dataset with 1000 iterations each to assess model detection accuracy.

### Example 2

### Study Subjects

Study subjects included individuals with a diagnosis of CRC, colorectal advanced adenomas (CAA), breast cancer (BC), lung cancer (LC), or pancreatic cancer (PC). CAA have traditionally been defined as adenomas >0.75 cm, with a villous component or high-grade dysplasia (Brenner et al. 2007, Gut, 56(11): 1585-9; Hassan et al., 2010, Aliment. Pharmacol. Ther., 31(2): 210-7). A recent systematic review found that a diameter >0.6 cm identifies 95% of individuals with a CAA, therefore, for the purposes of this study, CAA were defined as polyps >0.6 cm in diameter. Patients who had undergone a normal screening colonoscopy and had no malignancy or inflammatory condition served as a comparator “control” group. The “other” cancers that were chosen to be included in this study were selected because they frequently develop in individuals at ages similar to that at which CRC commonly occurs, and samples were readily available from the University of Louisville Surgical Biorepository and staged according to the American Joint Committee on Cancer TNM staging system (Edge et al., 2010, Ann. Surg. Oncol., 17(6): 1471-4). A total of 330 patients were included in this study. Patient demographics are shown in Table 1.

Peripheral blood was obtained from subjects in EDTA-vacutainers (BD, Franklin Lakes, N.J.). Plasma was immediately isolated from whole blood by centrifugation at 3500 rpm for 15 minutes as previously described (Kanaan et al., 2013, Ann. Surg., 258(3): 400-8) and then frozen at −80° C. for later use.

TABLE 1
Patient demographics

| | | | |
|---|---|---|---|
| | Training | Test Cohort | Validation |
| Variables | Cohort (n = 60) | (n = 120) | Cohort (n = 150) |

Controls

| | | | | |
|---|---|---|---|---|
| Age | Mean ± SD | 51 ± 11 | 40 ± 18 | 53 ± 20 |
| | Median (range) | 55 (27-63) | 29 (21-73) | 64 (22-74) |
| Gender | Male | 6 | 12 | 9 |
| | Female | 4 | 8 | 16 |
| Race | Caucasian | 10 | 16 | 23 |
| | African | 0 | 4 | 2 |
| | American | | | |
| | Asian | 0 | 0 | 0 |

Colorectal Cancer

| | | | | |
|---|---|---|---|---|
| Age | Mean ± SD | 55 ± 8 | 63 ± 8 | 59 ± 11 |
| | Median (range) | 55 (44-67) | 62 (47-76) | 59 (26-77) |
| Gender | Male | 5 | 10 | 12 |
| | Female | 5 | 10 | 13 |
| Race | Caucasian | 10 | 20 | 24 |
| | African | 0 | 0 | 1 |
| | American | | | |
| | Asian | 0 | 0 | 0 |

Colorectal Adenoma

| | | | | |
|---|---|---|---|---|
| Age | Mean ± SD | 64 ± 8 | 59 ± 12 | 63 ± 14 |
| | Median (range) | 63 (51-77) | 59 (38-79) | 64 (42-89) |
| Gender | Male | 5 | 6 | 8 |
| | Female | 5 | 14 | 17 |
| Race | Caucasian | 10 | 14 | 19 |
| | African | 0 | 4 | 5 |
| | American | | | |
| | Asian | 0 | 2 | 1 |

Breast Cancer

| | | | | |
|---|---|---|---|---|
| Age | Mean ± SD | 60 ± 10 | 53 ± 13 | 57 ± 9 |
| | Median (range) | 59 (48-79) | 53 (29-79) | 56 (39-77) |
| Gender | Male | 0 | 0 | 0 |
| | Female | 10 | 20 | 25 |
| Race | Caucasian | 10 | 18 | 24 |
| | African | 0 | 2 | 1 |
| | American | | | |
| | Asian | 0 | 0 | 0 |

Pancreatic Cancer

| | | | | |
|---|---|---|---|---|
| Age | Mean ± SD | 59 ± 6 | 65 ± 9 | 67 ± 9 |
| | Median (range) | 60 (49-69) | 62 (49-86) | 67 (51-86) |
| Gender | Male | 5 | 8 | 11 |
| | Female | 5 | 12 | 14 |
| Race | Caucasian | 10 | 20 | 23 |
| | African | 0 | 0 | 2 |
| | American | | | |
| | Asian | 0 | 0 | 0 |

Lung Cancer

| | | | | |
|---|---|---|---|---|
| Age | Mean ± SD | 58 ± 7 | 62 ± 10 | 58 ± 11 |
| | Median (range) | 60 (44-66) | 62 (44-81) | 56 (36-79) |
| Gender | Male | 5 | 11 | 11 |
| | Female | 5 | 9 | 14 |
| Race | Caucasian | 8 | 17 | 23 |
| | African | 2 | 3 | 2 |
| | American | | | |
| | Asian | 0 | 0 | 0 |

| American | Study Subjects | | | | | |
|---|---|---|---|---|---|---|
| Joint | | | | | Colorectal | |
| Committee | Colorectal | Breast | Pancreatic | Lung | Advanced | |
| on Cancer | Cancer | Cancer | Cancer | Cancer | Adenoma | Controls |
| Stage | (n = 55) | (n = 55) | (n = 55) | (n = 55) | (n = 55) | (n = 55) |
| 0 | 4 | 3 | 1 | 4 | Not | Not applicable |
| I | 8 | 21 | 5 | 19 | applicable | |
| II | 22 | 16 | 31 | 14 | >0.6 cm | |
| III | 4 | 9 | 6 | 11 | tubulovillous | |
| IV | 12 | 2 | 6 | 1 | or villous | |
| Not | 5 | 4 | 6 | 6 | | |
| available | | | | | | |

### Example 3

### Study Design

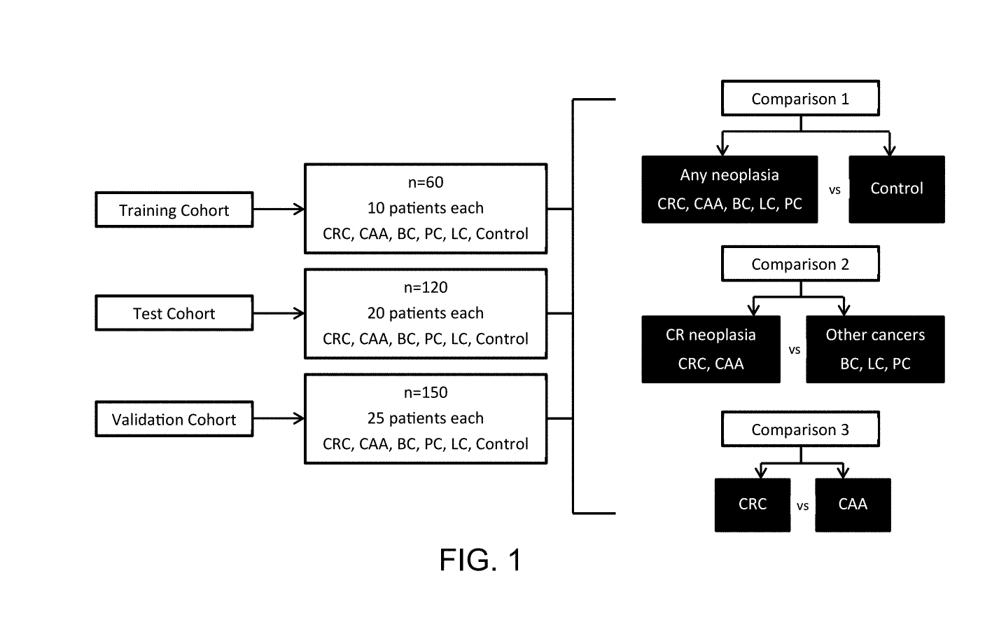

The study design is shown in FIG. 1. The study was performed in 3 stages as described below.

Stage 1—a “Training” cohort (n=60) or screening study to identify miRNA dysregulated in CRC and CAA (collectively referred to as colorectal neoplasia) as opposed to controls and other common cancers (breast, lung, pancreas);

Stage 2—a “Test” cohort (n=120) to confirm that the miRNA identified in Stage 1 were dysregulated in colorectal neoplasia as opposed to controls and other common cancers using single miRNA assays; and

Stage 3—a “Validation” cohort, (n=150) in which dysregulated miRNA expression was determined by single assay; this blinded data set was provided to the statisticians for determination of sample identity.

### Example 4

### Training Cohort

The Stage 1 “Training” cohort included sixty patients: 10 each with CRC, CAA, BC, LC, PC and 10 controls. Total RNA was extracted from plasma samples using the MIRNEASY® Serum/Plasma Isolation Kit (Qiagen, Valencia, Calif.). Total RNA quantity and purity of each sample were determined using a Nanodrop 2000 spectrophotometer (ThermoFisher Scientific®, Middlesex, Mass.). For each sample, 384 miRNAs were screened to identify dysregulated miRNA expression within each group as compared to controls (TAQMAN® Low Density Array (TLDA) human miRNA Card A, Life Technologies, Carlsbad, Calif.). Quantitative real-time polymerase chain reaction (qRT-PCR) was performed using a VIIA™ 7 Real-Time PCR System (ThermoFisher Scientific®, Middlesex, Mass.).

Data were analyzed as follows: “all neoplasia” versus control (Comparison 1), colorectal neoplasia versus “other” cancers (breast, lung and pancreas) (Comparison 2) and Colorectal Cancer (CRC) versus Colorectal Advanced Adenoma (CAA) (Comparison 3). See FIG. 1.

### Example 5

### Test Cohort

The Stage 2 “test” cohort included 120 samples: 20 patients in each group of CRC, CAA, BC, LC, PC and controls. Significantly dysregulated miRNAs identified from the Stage 1 “Training” cohort were validated using single miRNA assays. For miRNA single assay quantification, specific TAQMAN® miRNA primers for the dysregulated miRNAs and the two endogenous reference miRNA, RNU6 and miR-520d-5p (Rice et al., 2015, Surgery, 158(5): 1345-51) (Life Technologies, Carlsbad, Calif.) were then used to bind to complementary sequences on target cDNA during qRT-PCR. All reactions were run in duplicate. Nucleic acid quantification was performed using a Step-One Plus qRT-PCR system (Life Technologies, Carlsbad, Calif.).

### Example 6

### Validation Cohort

The Stage 3 “Validation” cohort included 150 samples: 25 samples from each group CRC, CAA, BC, LC, PC and controls, analyzed using the same single miRNA assay procedure as outlined for the Stage 2 “Test” cohort. These blinded data were then analyzed using a predictive model which had been generated using data from the “Test” cohort. This predictive model was then used to predict sample identity of the blinded data in the Stage 3 “Validation” cohort. Assessment for Comparisons 1, 2 and 3 was performed using the diagnostic miRNA panel to determine the accuracy of the prediction model.

### Example 7

### Statistical Consideration

Stage 1—Training Cohort

For the Stage 1 “Training” cohort, the miRNA expression of each sample group was compared to the miRNA expression of the control group by the comparative ΔCt analysis method, using RNU6 and miR-520d-5p as the endogenous reference genes (Rice et al., 2015, Surgery, 158(5): 1345-51). Statistical analysis using ANOVA identified significantly dysregulated miRNAs.

The method of Jung was used to identify about 5% of features to be significant at a false detection rate (FDR) of 5% with an adjusted alpha of 0.0038 (Jung et al., 2005, Bioinformatics, 21(14): 3097-104). With any two groups, with a minimum of n1=10 and n2=10 using a two sample t-test, a difference of at least 2.7-fold can be detected, using the common standard deviation at significance level of 0.0038 and a power of 80%. With respect to the choice of the number of miRNA in the panel, it was expected that no more than 10% of miRNAs would be differentially expressed between cases and controls after adjusting the p-values for multiple comparisons. Of these, in turn, one would not expect more than 0.5-3% of miRNAs to accurately identify cases and controls. Ten miRNAs and two reference miRNA genes were therefore chosen (approximately 3%) for further evaluation.

Stage 2—Test Cohort & Prediction Model for Sample Classification

For the Stage 2 “Test” cohort, Ct values for each miRNA in the panel were again analyzed using the comparative ΔCt method for each comparison. Similar to the “Training” cohort, comparisons 1, 2 and 3 as described herein were generated using data from the single miRNA assays, and receiver operating characteristic (ROC) curves were constructed and area-under-the-curve (AUC) were calculated (Carter et al., 2016, Surgery, 159(6): 1638-45) predictive models were fitted for each Comparison using the test dataset as follows, where pᵢ, p₂, and p₃ are the probabilities of a patient from the case group, which was all neoplasia for comparison 1, colorectal neoplasia for comparison 2, and CRC for comparison 3.

log(p₁)=1.48−0.29 ΔmiR374−0.36 ΔmiR29c−0.80 ΔmiR21−0.35 ΔmiR19a+1.42 ΔmiR150+1.04 ΔmiR346

log(p₂)=3.45−0.30 ΔmiR372+0.54 ΔmiR374−0.18 ΔmiR29c+0.06 ΔmiR21

log(p₃)=18.35−2.89 ΔmiR192−5.36 ΔmiR29c+1.11 ΔmiR21+12.33 ΔmiR19a−7.08 ΔmiR374−2.19 ΔmiR122

Using the “Test” cohort, a repeat sub-sampling validation method was employed using 50%, 60%, 70%, 80%, and 90% of the test dataset with 1000 iterations each in order to construct and subsequently assess the accuracy of the logistic prediction model (Pepe, 2003, Oxford University Press, USA; Rai et al. 2013, Open Access Med. Statistics, 3: 1-9). With this technique, the model was able to correctly identify controls from all other subjects with 88% accuracy and colorectal cancers from colorectal adenomas with 94% accuracy. This is based upon a 70% -30% training-test set combination; other results were similar.

Stage 3—Validation Cohort The logistic prediction models generated using the Test cohort data were used to predict sample identity in the validation cohort using the —Ct values of the 150 blinded samples. Four different methods were utilized: a normal-theory method with Unequal Variance (Parametric method) assuming unequal variances in the two groups; Kernel Density Estimates with Equal Bandwidth (Nonparametric method) using Normal kernel in the density estimation with Equal Bandwidth; k-Nearest Neighbors method (Nonparametric method) using 7 neighbors; and a multivariable logistic model to predict each sample's identity in the validation data set (Pepe, 2003, Oxford University Press, USA). Based on the prediction results and consideration of the sensitivity, specificity, and accuracy as binomial proportions, PROC FREQ (frequency procedures) was used to compute Agresti-Coull confidence limits for sensitivity, specificity and accuracy with their 95% confidence intervals for each analysis method and comparison (Agresti et al., 1998, The American Statistician, 52(2): 119-26). AUC with the 95% confidence interval was calculated by ROC analysis using data from the “Validation” cohort.

### Example 8

### Results

Stage 1—Training Cohort

Sixteen of 380 screened plasma miRNAs were significantly dysregulated when comparing all neoplasia (n=50) and controls (n=10) (Comparison 1) (p<0.05, FDR: 5%). Another sixteen miRNAs were significantly dysregulated when comparing colorectal neoplasia (CRC and CAA) (n=20) to other cancers (BC, PC, LC) (n=30) (Comparison 2) and a further six miRNAs were significantly dysregulated between colorectal cancer (n=10) and colorectal advanced adenoma (n=10) (Comparison 3). After reviewing the significantly dysregulated miRNA based on the adjusted p-value, AUC, fold change and biological significance, ten miRNAs and two endogenous reference miRNA were selected for further study (Table 2).

TABLE 2
miRNA panel of the 10 most significantly dysregulated miRNAs in
“Training” cohort after assessing p-value, fold change, AUC,
and biological significance

| | Adjusted p-value | | | Biological |
|---|---|---|---|---|
| Dysregulated | (False Discovery | Fold | | Significance |
| miRNA | Rate 5%) | Change | AUC | (reference) |
| miR-150 | <0.001 | 12.23 | 0.844 | Feng et al. |
| miR-193a | <0.001 | 9.087 | 0.835 | Zhang et al. |
| miR-374a | <0.001 | 0.001 | 0.879 | Wang et al. |
| miR-346 | <0.001 | 64.92 | 0.948 | Selth et al. |
| miR-29c | 0.001 | 0.241 | 0.811 | Kuo et al. |
| miR-19a | 0.002 | 0.186 | 0.775 | Zheng et al. |
| miR-192 | 0.002 | 0.303 | 0.834 | Chiang et al. |
| miR-21 | 0.006 | 0.559 | 0.794 | Kanaan et al. |
| miR-372 | 0.022 | 0.645 | 0.789 | Yamashita et al. |
| miR-122 | 0.037 | 1.388 | 0.75 | Kunte et al. |
| RNU6* | — | — | — | — |
| miR-520d-5p* | — | — | — | — |

*Endogenous reference miRNA

Stage 2—Test Cohort

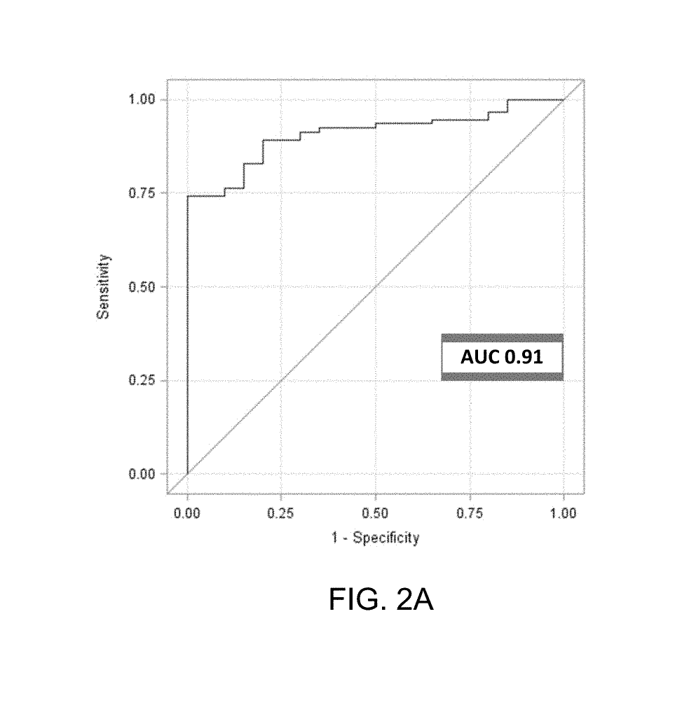

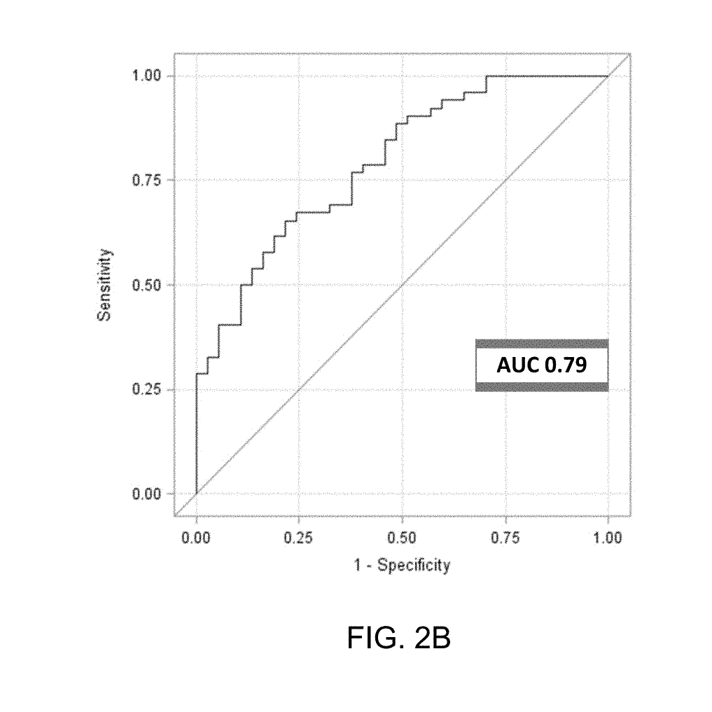

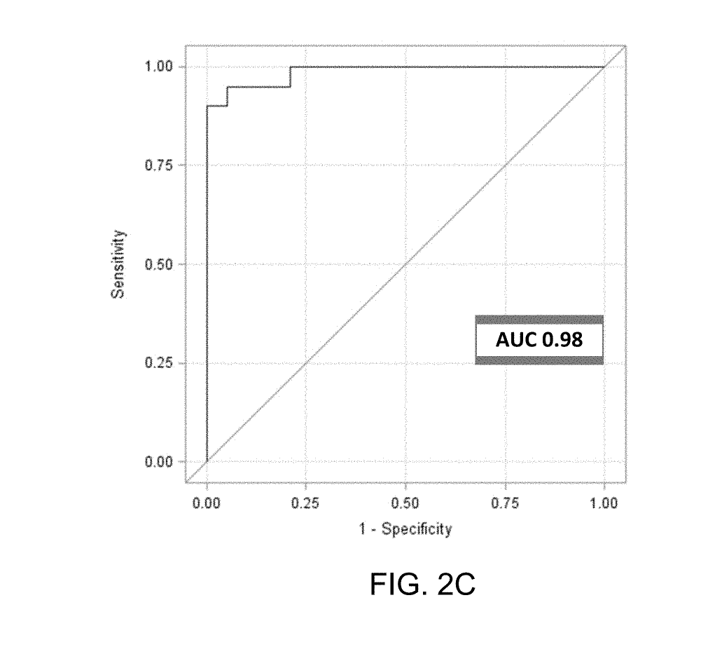

The ten selected miRNA were assessed utilizing a larger cohort (n=120). In Comparison 1 (n=100 vs. 20), four miRNAs, miR-21, miR-29c, miR-346 and miR-374a, demonstrated an AUC of 0.91 (95% CI: 0.85-0.96) in being able to differentiate patients with any type of neoplasia from controls. For Comparison 2 (n=40 vs. 60), miR-21, miR-29c, miR-372 and miR-374a demonstrated an AUC of 0.79 (95% CI: 0.70-0.88) in differentiating patients with colorectal neoplasia (CRC and CAA) from patients with other cancers (BC, LC and PC). In Comparison 3 (n=20 vs. 20), miR-29c, miR-122, miR-192 and miR-374a demonstrated an AUC of 0.98 (95% CI: 0.96-1.0) in being able to differentiate CRC from CAA (Table 3). ROC curves were generated to evaluate the diagnostic performance of the plasma miRNA in these three Comparisons (FIG. 2A, 2B & 2C).

TABLE 3
Panel of dysregulated miRNAs and AUC in “Test” cohort
for “All neoplasia” vs. “control”, “CR neoplasia” vs.
“Other cancers” and “CRC” vs. “CAA”

| | | Area Under the |
|---|---|---|
| Comparison | miRNA | Curve (95% CI) |
| Any neoplasia vs. control | miR-21 | 0.91 (0.85-0.96) |
| (n = 100 vs. 20) | miR-29c | |
| | miR-346 | |
| | miR-374a | |
| CR neoplasia vs. other cancers | miR-21 | 0.79 (0.70-0.88) |
| (n = 40 vs. 60) | miR-29c | |
| | miR-372 | |
| | miR-374a | |
| CRC vs. CAA | miR-29c | 0.98 (0.96-1.0) |
| (n = 20 vs. 20) | miR-122 | |
| | miR-192 | |
| | miR-374a | |

Stage 3—Validation Cohort

The predictive model developed using “Test” cohort data was then utilized on the blinded sample data of the validation cohort (n=150). In this cohort, for Comparison 1, using the predictive model with the four miRNAs, miR-21, miR-29c, miR-346 and miR-374a (n=125 vs. 25), correct prediction of sample identity between all neoplasia and control was achieved with 69-77% accuracy. In Comparison 2 (n=50 vs. 75), miR-21, miR-29c, miR-372 and miR-374a, predicted sample identity with 67-76% accuracy between CR neoplasia and other cancers. Finally, in Comparison 3 (n=25 vs 25), miR-29c, miR-122, miR-192 and miR-374a predicted sample identity with 86-90% accuracy between CRC and CAA. Table 4 shows the individual sensitivity, specificity, AUC and accuracy for each Comparison.

TABLE 4
Sensitivity, specificity, AUC & accuracy of miRNA panels for “All neoplasia” vs. “control”,
“CR neoplasia” vs. “Other cancers” and “CRC” vs. “CAA” in the “Validation” cohort

| | Analysis | Sensitivity | Specificity | AUC | Accuracy | Accuracy |
|---|---|---|---|---|---|---|
| Comparison | Method* | (95% CI) | (95% CI) | (95% CI) | (95% CI) | Range (%) |
| All neoplasia vs. | NorUnEqual | 81.6 (73.8-87.5) | 32.0 (17.1-51.7) | 0.62 (0.50-0.74) | 73.3 (65.7-79.8) | 68.7-77.3 |
| Controls | KDEEqual | 85.6 (78.3-90.8) | 36.0 (20.2-55.6) | | 77.3 (70.0-83.3) | |
| (n = 125 vs. 25) | KNN7 | 72.0 (63.5-79.2) | 52.0 (33.5-70.0) | | 68.7 (60.8-75.6) | |
| | Multivariable | 84.0 (76.5-89.5) | 28.0 (14.1-47.8) | | 74.7 (67.1-81.0) | |
| CR Neoplasia | NorUnEqual | 69.4 (55.4-80.6) | 80.0 (69.5-87.6) | 0.76 (0.68-0.84) | 75.8 (67.5-82.5) | 66.9-75.8 |
| vs. Other | KDEEqual | 63.3 (49.2-75.4) | 80.0 (69.5-87.6) | | 73.4 (65.0-80.4) | |
| cancers | KNN7 | 63.3 (49.2-75.4) | 77.3 (66.6-85.4) | | 71.8 (63.3-79.0) | |
| (n = 50 vs. 75) | Multivariable | 65.3 (51.3-77.1) | 68.0 (56.8-77.5) | | 66.9 (58.2-74.6) | |
| CRC vs. CAA | NorUnEqual | 76.0 (56.2-88.8) | 100.0 (84.2-100.0) | 0.98 (0.94-1.0) | 88.0 (75.8-94.8) | 86.0-90.0 |
| (n = 25 vs. 25) | KDEEqual | 72.0 (52.2-85.9) | 100.0 (84.2-100.0) | | 86.0 (73.5-93.4) | |
| | KNN7 | 80.0 (60.4-91.6) | 100.0 (84.2-100.0) | | 90.0 (78.2-96.1) | |
| | Multivariable | 80.0 (60.4-91.6) | 100.0 (84.2-100.0) | | 90.0 (78.2-96.1) | |

*NorUnEqual: Normal-theory method with Unequal Variance assuming unequal variances in the two groups;
KDEEqual: Kernel Density Estimates with Equal Bandwidth using Normal kernel in the density estimation with Equal Bandwidth;
KNN7: k-Nearest Neighbors method using 7 neighbors;
Multivariable: Multivariable logistic model.

Seven miRNAs (miR-21, miR-29c, miR-122, miR-192, miR-346, miR-372, miR-374a) were selected based upon p-value, area-under-the-curve (AUC), fold-change, and biological plausibility. AUC (±95% CI) for “Test” cohort Comparisons were 0.91 (0.85-0.96), 0.79 (0.70-0.88) and 0.98 (0.96-1.0), respectively. The mathematical model described herein predicted blinded sample identity with 69-77% accuracy between all neoplasia and controls, 67-76% accuracy between colorectal neoplasia and other cancers, and 86-90% accuracy between colorectal cancer and colorectal adenoma. Thus, the plasma miRNA assay and prediction model described herein differentiates colorectal neoplasia from patients with other neoplasms and from controls with higher sensitivity and specificity compared to current clinical standards.

### Example 9

### Refinement of Models

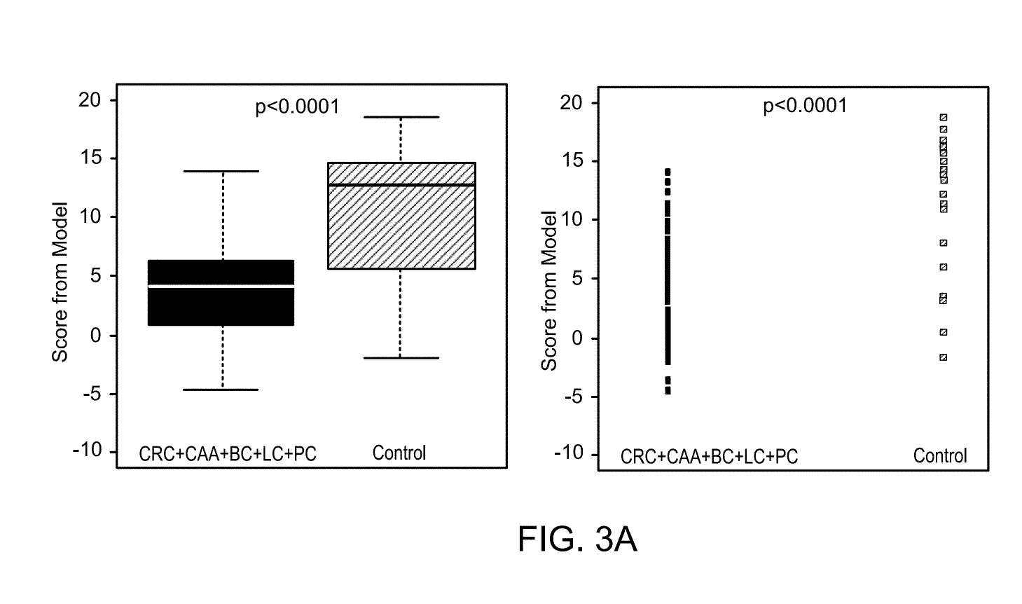

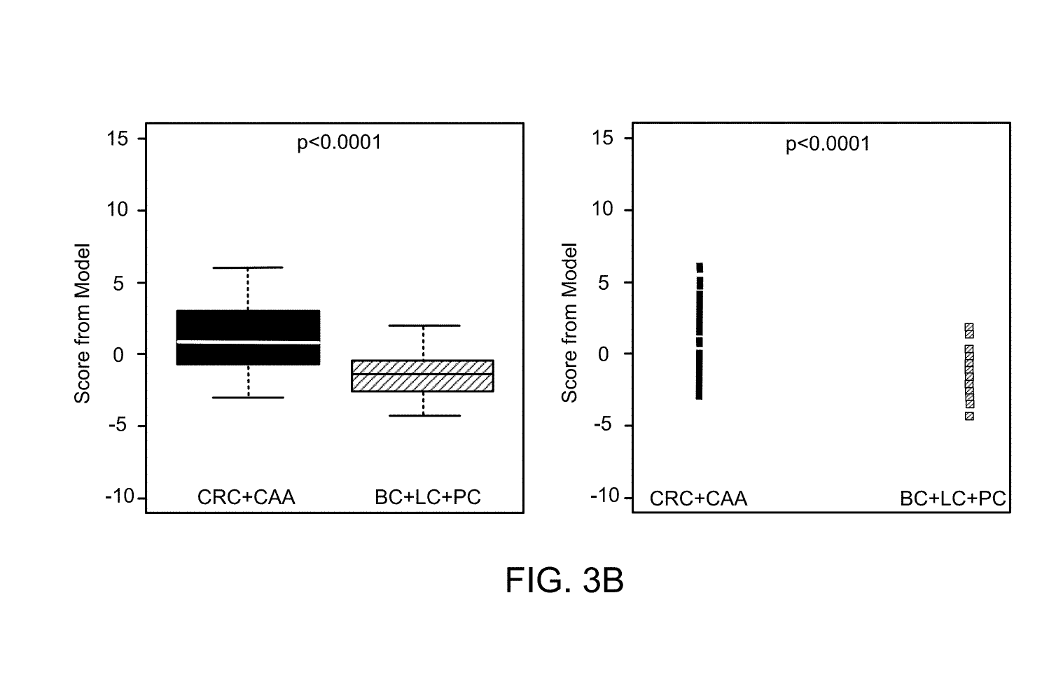

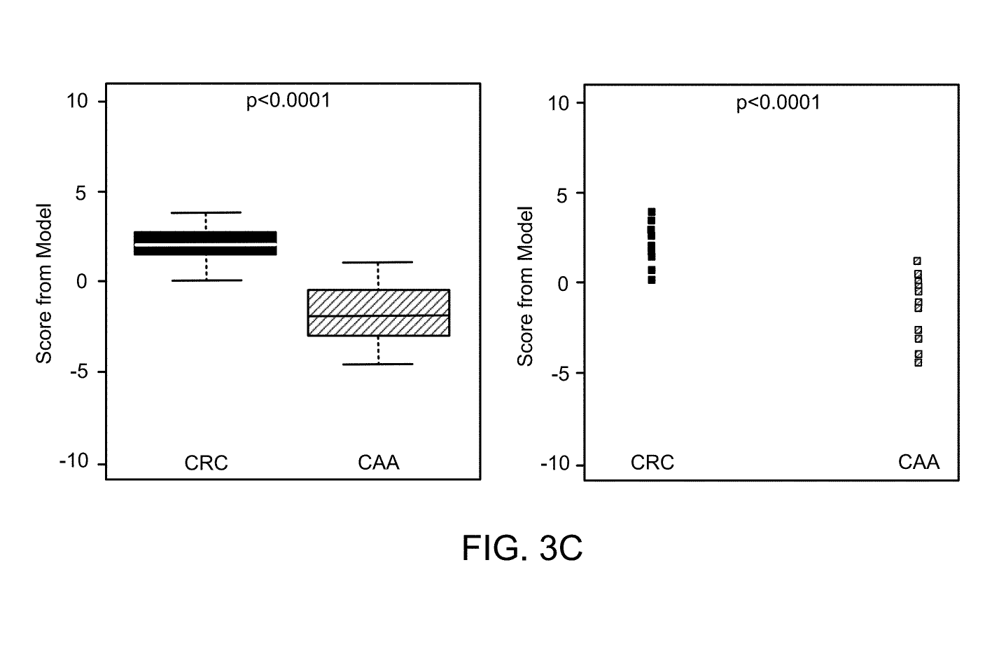

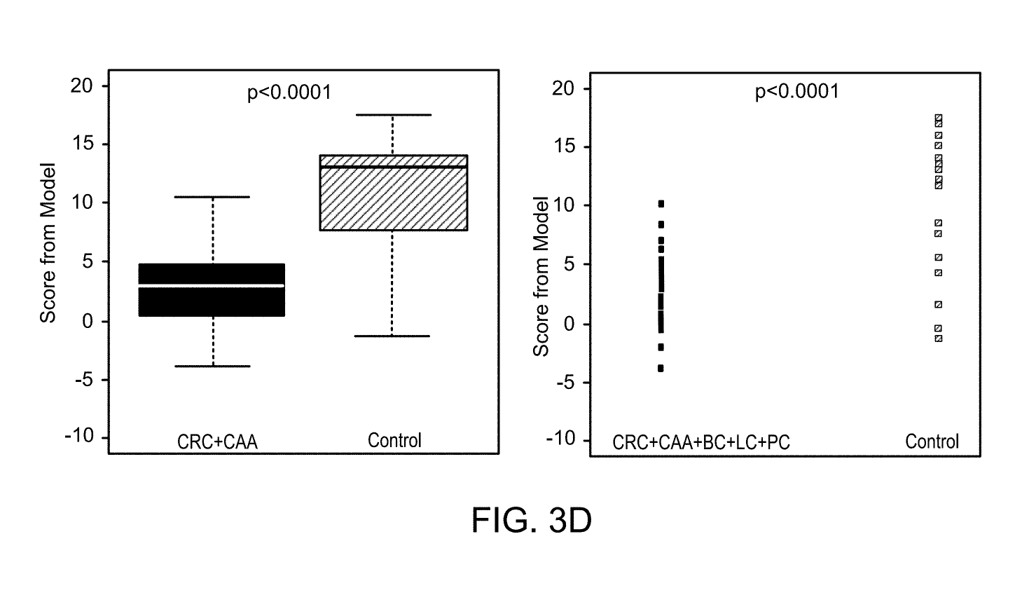

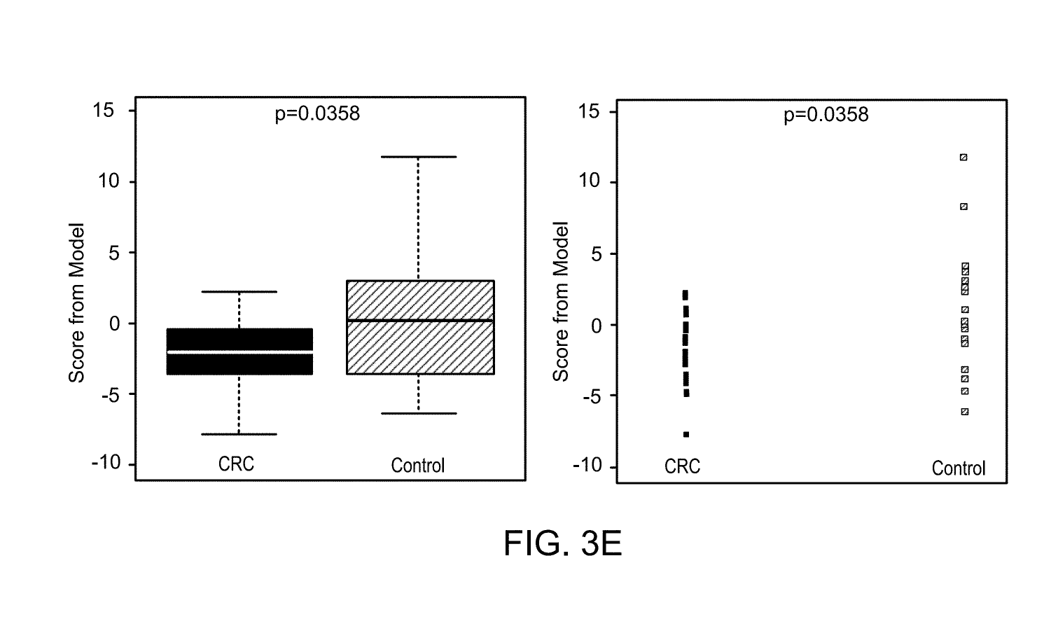

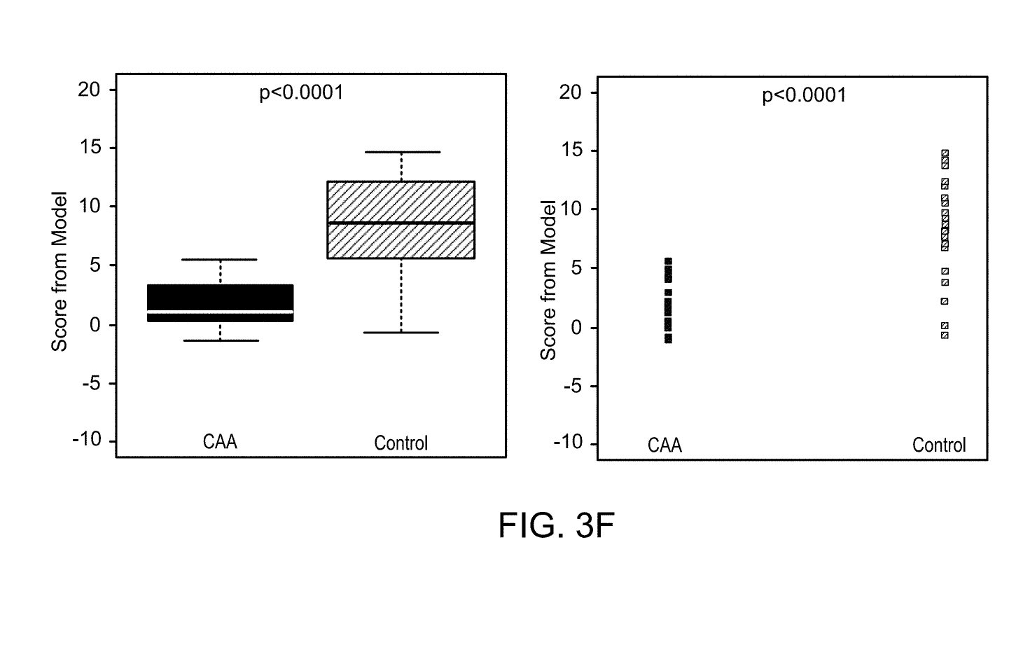

For Comparison 1, FIG. 3A shows the prediction scores for comparing CRC, CAA, breast cancer (BC), lung cancer (LC), and pancreas cancer (PC) vs. Controls in the Test cohort. The following shows the prediction model for comparing CRC, CAA, BC, LC, and PC vs. Controls, and Table 5 shows the corresponding prediction parameters.

$$log ( \frac{p}{1 - p} ) = 2.9 \text{?} - 1.703 \times miR 21 + 1.107 \times miR 193 \text{?} - 5 p + 2.200 \times miR 84 \text{?} - 1.124 \times miR \text{?} 72$$

$$\text{?} \text{indicates text missing or illegible when filed}$$

TABLE 5
Prediction Parameters for Comparing CRC, CAA, Breast Cancer,
Lung Cancer, Pancreas Cancer vs. Control in the Test Cohort

| Com- | AUC | Sensitivity | Specificity | Accuracy |
|---|---|---|---|---|
| parison | (95% CI) | (95% CI) | (95% CI) | (95% CI) |
| 1) | 0.951 | 0.888 | 0.900 | 0.890 |
| CRC + | (0.909-0.993) | (0.804-0.94) | (0.687-0.984) | (0.816-0.937) |
| CAA + | | | | |
| BC + | | | | |
| LC + | | | | |
| PC vs. | | | | |
| CON | | | | |

For Comparison 2, FIG. 3B shows the prediction scores for comparing CAA+CRC vs. breast cancer, lung cancer, and pancreas cancer in the Test cohort. The following shows the prediction model for comparing CAA+CRC vs. breast cancer, lung cancer and pancreas cancer in the Test cohort, and Table 6 shows the corresponding prediction parameters.

$$log ( \frac{p}{1 - p} ) = 6.026 + 0. \text{?} \times miR 374 - 0.928 \times miR 19 \text{?} - 5 p + 0.64 \text{?} \times miR \text{?} 46 - 0.681 \times miR \text{?} 72 + 0. \text{?} 24 \times miR 122$$

$$\text{?} \text{indicates text missing or illegible when filed}$$

TABLE 6
Prediction Parameters for Comparing CAA + CRC vs. Breast Cancer, Lung Cancer,
Pancreas Cancer in the Test Cohort

| | AUC | Sensitivity | Specificity | Accuracy |
|---|---|---|---|---|
| Comparison | (95% CI) | (95% CI) | (95% CI) | (95% CI) |
| 2) CRC + CAA vs. BC + LC + PC | 0.879 | 0.811 | 0.800 | 0.804 |
| | (0.811-0.946) | (0.655-0.908) | (0.675-0.886) | (0.711-0.873) |

For Comparison 3, FIG. 3C shows the prediction scores for comparing CRC vs. CAA in the Test cohort. The following shows the prediction model for comparing CAA vs. CRC in the Test cohort, and Table 7 shows the corresponding prediction parameters.

$$log ( \frac{p}{1 - p} ) = - 2.779 - 0. \text{?} \times miR 374$$

$$\text{?} \text{indicates text missing or illegible when filed}$$

TABLE 7
Prediction Parameters for Comparing CAA vs. CRC in the Test Cohort

| Com- | AUC | Sensitivity | Specificity | Accuracy |
|---|---|---|---|---|
| parison | (95% CI) | (95% CI) | (95% CI) | (95% CI) |
| 3) CRC | 0.916 | 0.842 | 0.950 | 0.897 |
| vs. CAA | (0.822-1.0) | (0.616-0.953) | (0.746-1.0) | (0.758-0.965) |

For Comparison 4, FIG. 3D shows the prediction scores for comparing CRC and CAA vs. Control in the Test cohort. The following shows the prediction model for comparing CAA+CRC vs. Control in the Test cohort, and Table 8 shows the corresponding prediction parameters.

$$log ( \frac{p}{1 - p} ) = 6.4 \text{?} - 1.186 \times miR 21 + 1.000 \times miR 180 + 1.961 \times miR \text{?} 46 - 1.224 \times miR \text{?} 72$$

$$\text{?} \text{indicates text missing or illegible when filed}$$

TABLE 8
Prediction Parameters for Comparing CAA + CRC vs. Control in the Test Cohort

| | AUC | Sensitivity | Specificity | Accuracy |
|---|---|---|---|---|
| Comparison | (95% CI) | (95% CI) | (95% CI) | (95% CI) |
| CRC + CAA vs. CON | 0.926 | 0.919 | 0.800 | 0.877 |
| | (0.858-0.993) | (0.780-0.979) | (0.578-0.925) | (0.764-0.942) |

For Comparison 5, FIG. 3E shows the prediction scores for comparing CRC vs. Control in the Test data set. The following shows the prediction model for comparing CRC vs. Control in the Test cohort, and Table 9 shows the corresponding prediction parameters.

$$log ( \frac{p}{1 - p} ) = 4.727 - 1. \text{?} 94 \times miR 192 - 1. \text{?} 10 \times miR 1 \text{?} 0 + 1.7 \text{?} \times miR \text{?}$$

$$\text{?} \text{indicates text missing or illegible when filed}$$

TABLE 9
Prediction Parameters for Comparing CRC vs. Control in the Test Cohort

| Com- | AUC | Sensitivity | Specificity | Accuracy |
|---|---|---|---|---|
| parison | (95% CI) | (95% CI) | (95% CI) | (95% CI) |
| CRC vs. | 0.953 | 0.895 | 1.000 | 0.949 |
| CON | (0.877-1.0) | (0.674-0.983) | (0.810-1.0) | (0.822-0.995) |

For Comparison 6, FIG. 3F shows the prediction scores for comparing CAA vs.

Control in the Test cohort. The following shows the prediction model for comparing CAA vs. Control in the Test cohort, and Table 10 shows the corresponding prediction parameters.

$$log ( \frac{p}{1 - p} ) = \text{?} 00 - 0.6281 ⋆ miR 21 + 0. \text{?} \times miR \text{?} 74 - 1.297 ⋆ miR \text{?} 72 + 0. \text{?} ⋆ miR 122$$

$$\text{?} \text{indicates text missing or illegible when filed}$$

TABLE 10
Prediction Parameters for Comparing CAA vs. Control in the Test Cohort

| Com- | | | | |
|---|---|---|---|---|
| pari- | AUC | Sensitivity | Specificity | Accuracy |
| son | (95% CI) | (95% CI) | (95% CI) | (95% CI) |
| CAA | 0.889 | 0.789 | 0.850 | 0.821 |
| vs. | (0.791-0.988) | (0.561-0.920) | (0.631-0.956) | (0.670-0.913) |
| CON | | | | |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1-10. (canceled)

11. A method of determining a treatment for a subject having a neoplasia, the method comprising:
(a) determining expression levels of miRNAs comprising miR-21, miR-193a, miR-346, and miR-372 in a biological sample from the subject;
(b) applying the following algorithm to the expression levels determined in step (a):
$$log ( \frac{p}{1 - p} ) = 2.950 - 1.706 \times miR 21 + 1.107 \times miR 193 a - 5 p + 2.200 \times miR 346 - 1.124 \times miR 372 ;$$
and
(c) administering a treatment to the subject based upon the results of the algorithm of step (b).

12. The method of claim 11, wherein the neoplasia is selected from CRC, CAA, BC, LC or PC.

13. The method of claim 11, wherein the method is computer-implemented.

14. The method of claim 12, wherein the method is computer-implemented.

15. A method of selecting a treatment regimen for a subject having a neoplasia selected from CAA or CRC, the method comprising:
(a) determining expression levels of miRNAs comprising miR-122, miR193a, miR-346, miR-372 and miR-374 in a biological sample from the subject;
(b) applying the following algorithm to the expression levels determined in step (a):
$$log ( \frac{p}{1 - p} ) = 6.026 + 0.513 \times miR 374 - 0.928 \times miR 193 a - 5 p + 0.649 \times miR 346 - 0.681 \times miR 372 + 0.524 \times miR 122 ;$$
and
(c) selecting a treatment regimen for the subject based upon the results of the algorithm of step (b).

16. The method of claim 15, wherein the method is computer-implemented.

17. A method of selecting a treatment regimen for a subject having a neoplasia selected from CRC or CAA, the method comprising:
(a) determining expression level of miR-374 in a biological sample from the subject;
(b) applying the following algorithm to the expression levels determined in step (a):
$$log ( \frac{p}{1 - p} ) = - 5.779 - 0.915 \times miR 374 ;$$
and
(c) selecting a treatment regimen for the subject based upon the results of the algorithm of step (b).

18. The method of claim 17, wherein the method is computer-implemented.

19. A method of treating a subject having a neoplasia, the method comprising:
(a) determining expression levels of miRNAs comprising miR-21, miR-150, miR-346, and miR-372 in a biological sample from the subject;
(b) applying the following algorithm to the expression levels determined in step (a):
$$log ( \frac{p}{1 - p} ) = 6.435 - 1.156 \times miR 21 + 1.000 \times miR 150 + 1.961 \times miR 346 - 1.224 \times miR 372 ;$$
and
(c) administering a treatment to the subject based upon the results of the algorithm of step (b).

20. The method of claim 19, wherein the neoplasia is CRC or CAA.

21. The method of claim 19, wherein the method is computer-implemented.

22. The method of claim 20, wherein the method is computer-implemented.

23. A method of treating a subject having CRC, the method comprising:
(a) determining expression levels of miRNAs comprising miR-150, miR-192, and miR-346, in a biological sample from the subject;
(b) applying the following algorithm to the expression levels determined in step (a):
$$log ( \frac{p}{1 - p} ) = 4.727 - 1.694 \times miR 192 + 1.310 \times miR 150 + 1.758 \times miR 346 ;$$
and
(c) administering a treatment to the subject based upon the results of the algorithm of step (b).

24. The method of claim 23, wherein the method is computer-implemented.

25. A method of treating a subject having CAA, the method comprising:
(a) determining expression levels of miRNAs comprising miR-21, miR-122, miR-372 and miR-374 in a biological sample from the subject;
(b) applying the following algorithm to the expression levels determined in step (a):
$$log ( \frac{p}{1 - p} ) = 3.800 - 0.623 ⋆ miR 21 + 0.899 ⋆ miR 374 - 1.297 ⋆ miR 372 + 0. \text{?} ⋆ miR 122 ;$$
$$\text{?} \text{indicates text missing or illegible when filed}$$
and
(c) administering a treatment to the subject based upon the results of the algorithm of step (b).

26. The method of claim 25, wherein the method is computer-implemented.
